(19) United States
(12) Patent Application Publication
(10) Pub. No.: US 2024/0307308 A1
(43) Pub. Date: Sep. 19, 2024

(54) PORPHYRIN NANOVESICLE WITH FATTY ACID CONJUGATE

(71) Applicant: UNIVERSITY HEALTH NETWORK, Toronto (CA)

(72) Inventors: Gang ZHENG, Toronto (CA); Juan CHEN, Toronto (CA); Tiffany Thanh-Uyen HO, Toronto (CA); Jiachuan BU, Toronto (CA); Lili DING, Richmond Hill (CA)

(21) Appl. No.: 18/578,888

(22) PCT Filed: Jul. 12, 2022
(86) PCT No.: PCT/CA2022/051087

§ 371 Date: Jan. 12, 2024

Related U.S. Application Data
(60) Provisional application No. 63/221,212, filed on Jul. 13, 2021.

(51) Int. Cl. A61K 9/127; A61K 41/00; A61K 51/04; A61K 51/12

(52) U.S. Cl. CPC A61K 9/1272; A61K 41/0071; A61K 51/0497; A61K 51/1234

(57) ABSTRACT

There is described herein a bilayer nanovesicle comprising porphyrin-phospholipid conjugate and a chelator-fatty acid conjugate; wherein the chelator-fatty acid conjugate comprises an aminopolycarboxylic acid conjugated to a single chain fatty acid; and the porphyrin-phospholipid conjugate comprises one porphyrin, porphyrin derivative or porphyrin analog covalently attached to a lipid side chain, preferably at the sn-1 or the sn-2 position, of one phospholipid.

### RELATED APPLICATIONS

This application claims priority to U.S. Provisional Patent Application No. 63/221,212 filed on Jul. 13, 2021, incorporated herein by reference in its entirety.

### FIELD OF THE INVENTION

The invention relates to nanovesicles comprising porphyrin and a fatty acid conjugate, and methods of use for said nanovesicles.

### BACKGROUND OF THE INVENTION

Photodynamic therapy (PDT) involves the combination of non-toxic photosensitizers and light of an appropriate wavelength, which in the presence of oxygen allows for the generation of reactive oxygen species for cell death and tissue destruction. PDT is attractive as a cancer therapy because of its dual selectivity; preferential accumulation of the photosensitizer within the tumor tissue and spatially focused light on the targeted area, which confines photosensitizer activation to the localized region. PDT also offers many advantages over conventional cancer therapies as it's minimally invasive, causes little to no scarring, and the same target site can be treated multiple times if needed¹⁻³. Most photosensitizers are administered systemically at the clinical level, where their accumulation in the tumor site directs the drug to light interval (DLI) for treatment; short DLI is used to target the tumor vasculature for vascular PDT, and prolonged DLI is used for optimal distribution of photosensitizers at the cellular level (cellular-PDT)⁴.

Porphyrins are ubiquitous compounds present in nature and are involved in numerous biological processes, such as photosynthesis (chlorophyll) and oxygen transport (heme). Porphyrins are tetrapyrrole macrocycles that are interconnected by methine bridges into a ring-like structure that confers their stability and optical properties. Porphyrins and their derivatives have been clinically approved as efficacious PDT agents for treatment in lung, esophageal, bile duct, bladder, ovarian, and cervical cancers⁵⁻⁹. However, many porphyrin-based photosensitizers used clinically possess various defects that limit their applications, such as low chemical purity, weak tissue penetration, subpar tumor accumulation, and excessive lipophilicity that cause them to aggregate in biological fluids, leading to dark toxicity and prolonged light sensitivity¹⁰.

Porphysomes are self-assembled liposome-like bilayered nanovesicles (˜100 nm diameter) composed of porphyrin-lipids that are well-characterized and biocompatible organic molecules and enzymatically biodegradable in vivo. Administration of porphyrin-lipid at doses of 1000 mg/kg elicited minimal acute toxicities in mice^{11, 12}. Porphysomes contain a high density of porphyrin photosensitizers (>80,000 porphyrins per particle) that are tightly packed within the nanostructure, resulting in ‘super’-quenching in fluorescence and singlet oxygen generation¹¹, which can effectively convert light of specific wavelengths to heat with extremely high efficiency, giving them ideal photothermal and photoacoustic properties that are unprecedented in organic nanoparticles. Upon porphysome nanostructure dissociation, fluorescence and photoreactivity of free porphyrins are restored to enable low background fluorescence imaging and activatable photodynamic therapy. In addition, Radioisotopes (e.g., ⁶⁴Cu) or metals (e.g., manganese) can be robustly chelated into porphyrin ring to allow positron emission tomography (PET) and magnetic resonance imaging (MRI), respectively¹³⁻¹⁵. As a result, the simple yet all-encompassing nature of the porphysome represents a new paradigm in nanomedicine design for multimodal imaging and therapeutics.

Porphysomes are potentially well-suited for selective PDT as they deliver large quantities of porphyrin in photoreactivity quenched condition, preferentially accumulate and be eventually activated in tumors, and the activation could be tracked by unquenching of fluorescence¹¹. However, conventional porphysomes' PDT application is limited by their slow intracellular uptake and uncontrolled/unpredictable activation in cancer cells, which raises the challenges on defining optimal DLI for efficacious PDT that highly depends on the amount of active photosensitizers localized within the target site. Therefore, there is a need to find an active approach to control and trigger the accumulation and activation of porphysome into tumors for effective PDT. For example, we have reported that inclusion of active targeting, such as folate-conjugated PS, could significantly enhance porphysome intracellular uptake in KB cells after 3 and 24 h incubation, which resulted in significantly enhanced PDT in mice tumor model¹⁶. Ethylenediaminetetraacetic acid (EDTA) has been approved clinically to treat heavy metal intoxication since 1953^{17, 18}. It has also been investigated in various antimicrobial studies, as chelation of divalent metal cations to increase the cell wall membrane permeability of gram-negative bacteria to exogenous agents¹⁹. In addition, EDTA is considered an absorption enhancer that promotes the penetration of drugs, proteins, and peptides through the corneal, nasal, and intestinal epithelium²⁰. It has been used to improve paracellular permeability by temporarily loosening the tight junctions between adjacent epithelial cells²⁰. Recently, EDTA chelation therapy has been also studied for the treatment of cardiovascular disease, neurodegenerative disease, and cancer²¹⁻²⁵.

### SUMMARY OF THE INVENTION

In an aspect, there is provided a bilayer nanovesicle comprising porphyrin-phospholipid conjugate and a chelator-fatty acid conjugate; wherein the chelator-fatty acid conjugate comprises an aminopolycarboxylic acid conjugated to a single chain fatty acid; and the porphyrin-phospholipid conjugate comprises one porphyrin, porphyrin derivative or porphyrin analog covalently attached to a lipid side chain, preferably at the sn-1 or the sn-2 position, of one phospholipid.

In an aspect, there is provided a composition comprising the bilayer nanovesicles described herein in a buffer.

In an aspect, there is provided a method of performing photodynamic therapy to a target area on a subject, comprising: providing the composition described herein; administering the composition to the subject; and irradiating the target area with light of a wavelength that excites the composition to create radicals and/or reactive oxygen species.

In an aspect, there is provided a method of imaging a target area in a subject, comprising providing the composition described herein; administering the composition to the subject; and measuring and/or detecting fluorescence or photoacoustic signal at the target area.

In an aspect, there is provided a method of delivering a radioisotope to a subject comprising: providing the composition described herein, wherein the bilayer nanovesicle has a radioisotope chelated therein; and administering the composition to the subject.

In an aspect, there is provided a use of the composition described herein for performing photodynamic therapy.

In an aspect, there is provided a use of the composition described herein for performing imaging.

In an aspect, there is provided a use of the composition described herein for delivering a radioisotope to a subject.

### BRIEF DESCRIPTION OF FIGURES

These and other features of the preferred embodiments of the invention will become more apparent in the following detailed description in which reference is made to the appended drawings wherein:

FIG. 1 shows scheme EDTA-lipid-based new porphysome (eNPS).

FIG. 2 shows A) A representative TEM image of NPS; B) Absorption of intact vs. disrupted NPS and C) Fluorescence generation of intact NPS vs. disrupted NPS.

FIG. 3 shows eNPS intracellular uptake compared to PS was detected by A) fluorescence microscopy image and B) quantitative fluorescence measurement after cell extraction. A two-way ANOVA with Bonferroni correction was used to determine significant differences, *p<0.05, **p<0.01, ***p<0.001, ****p<0.0001.

FIG. 4 shows eNPS uptake by KB epithelium cells vs. normal fibroblast cells (NFB) under fluorescence microscopy imaging.

FIG. 5 shows A) chemical structure of DTPA-lipid W/O Gd³⁺ chelation and their corresponding TEM images; B) Comparing intracellular uptake of dNPS, eNPS and PS by B) fluorescence microscopy imaging (Scale bar=20 μm) and C) quantitative cell uptake analysis. A two-way ANOVA with Bonferroni correction was used to determine significant differences, *p<0.05, **p<0.01, ***p<0.0001 .

FIG. 6 shows effect of Metal-chelation on eNPS and dNPS uptake. A) Fluorescence microscopy imaging reveal that preincubation with 1 mM of Ca²⁺ or Mg²⁺ did not affect eNPS-mediated rapid uptake in KB cell; B) Gd³⁺ chelation did not affect dNPS uptake (DPS vs. DPS(Gd). A two-way ANOVA with Bonferroni correction was used to determine significant differences, *p<0.05.

FIG. 7 shows investigation of lipid chain effect on NPS uptake by replacing single fatty acid DTPA-lipid with various double fatty acid DTPA-lipids. A) chemical structure of various DTPA-lipids and the TEM image of corresponding NPS formed by these DTPA-lipiods; B) intracellular uptake compared with dNPS.

FIG. 8 shows A) Normalized cellular uptake (n=3) of eNPS, LC-eNPS to PS, *p<0.05, **p<0.0001, two-way ANOVA; B) Serum stability of eNPS (n=3), LC-eNPS (n=3), and PS (n=3) in 50 vol % FBS tracked by fluorescence quenching efficiency.

FIG. 9 shows temperature and energy-dependent uptake of eNPS and dNPS in KB cells. Fluorescence microscopy images of KB cells incubated with transferrin (Tfn), eNPS, and dNPS: A) at different temperature for 3 hours; B) co-incubated with NaN3 and 2-DG for 1 hour. Scale bar=20 μm.

FIG. 10 shows subcellular localization of eNPS and dNPS was examined on KB cells under confocal microscopy by co-incubated with A) Mitotracker and B) Lysotracker.

FIG. 11 shows in vitro PDT evaluation on KB cells after incubation with 5 μM of eNPS, PS, and LC-eNPS for different time duration (3, 6 or 24 h), respectively, and then received different dose of light irradiation. treatment varied light dose. The cell viability was evaluated by alamarblue assay.

FIG. 12 shows in vivo fluorescence imaging of eNPS vs. PS on KB subcutaneous mouse model.

FIG. 13 shows fluorescence imaging of eNPS vs. PS on a hamster cheek carcinogenesis model by a NOVADAQ Pinpoint system. A) time-dependent fluorescence images; and B) tumor florescence intensity tracking profile with time in tumor for eNPS and PS.

FIG. 14 shows EDTA-hexadecylamide (EDTA-lipid) synthesis.

FIG. 15 shows the fluorescence imaging of cells immediately and at 3 h and 18 h after removing the incubation medium containing PS, eNPS2 and eNPS3 (1 μM) at 3 h, 6 h and 24 h post incubation. The results did not appear signal enhancement upon post-incubation, which suggests that eNPS undergo rapid activation within the intracellular environment.

FIG. 16 shows co-incubation of high concentration of EDTA enables increased uptake of PS. The result demonstrated that unlike eNPS-mediated delivery enhancement (FIG. 4) (5 μM eNPS contains no more than 7 μM of EDTA-lipid), 1 mM of free EDTA exhibited negligible effect on PS uptake

FIG. 17 shows for incorporation EDTA-lipid in liposome formulation doped 1 mol % porphyrin lipid for fluorescence imaging, the resultant 1% pyro-eNPS (1 μM based on pyro concentration) exhibited similar rapid uptake as eNPS did in incorporation EDTA-lipid in FIG. 4. EDTA-lipid plays same role on triggering nanoparticle rapid uptake when incorporation EDTA-lipid in liposome formulation containing 1 mol % porphyrin lipid.

FIG. 18 shows a comparison of in vivo PDT efficacy of eNPS, LC-eNPS, and PS at varied drug-light-interval (DLI). Both eNPS and LC-eNPS (3 h DLI) demonstrated the highest in vivo PDT efficacy compared to PS. A two-way ANOVA with a Tukey post-hoc test was used to determine significant difference between groups (*p<0.05, **p<0.01, ***p<0.0001).

FIG. 19 shows a comparison of in vivo fluorescence activation of eNPS, LC-eNPS, and PS at various timepoints post-injection. Both eNPS and LC-eNPS demonstrated the highest in vivo fluorescence activation at all timepoints compared to PS.

### DETAILED DESCRIPTION

In the following description, numerous specific details are set forth to provide a thorough understanding of the invention. However, it is understood that the invention may be practiced without these specific details.

In this study, we designed a non-toxic EDTA-hexadecylamide conjugate (EDTA-lipid) for porphyrin nanovesicle construction to create the next generation porphysome (NPS) that exhibited much improved porphysome intracellular uptake while maintaining good serum stability. When NPS contains EDTA-lipid up to 50 mol % of total lipid contents in porphysome, an impressive 20-fold enhancement in cancer cell internalization was achieved at 24 h incubation, which sequentially resulted in drastically increased PDT efficacy (7% cell viability) compared to conventional porphysome (97% cell viability) at 5 μM concentration. This NPS platform can also be extended from EDTA-lipid to diethylenetriaminepentaacetic acid-hexadecylamide conjugate (DTPA-lipid). As a result, different radioisotope (e.g., ^{99m}Tc) or metal ions (e.g., gadolinium) can be robustly chelated into DTPA to compliment the intrinsic metal chelation properties of porphysomes. Interestingly, it was found that the main mechanism of EDTA-lipid or DTPA-lipid-enhanced porphysome intracellular delivery was not dependent on Ca²⁺ or Mg²⁺ chelation as what one would predict for classic EDTA-enhanced cell membrane permeability. This finding was supported by multiple lines of evidence: 1) Unlike NPS-mediated enhanced uptake in epithelium cells, co-incubation of PS with 1 mM concentration of EDTA did not enhance intercellular uptake of porphysome; 2) EDTA-lipid-based NPS (eNPS) after pre-incubation with high concentration of Ca²⁺ and Mg²⁺ did not diminish eNPS-enhanced delivery; 3) DTPA-lipid-contained NPS (dNPS) with and without Gd³⁺ chelation, both showed similar enhanced uptake as eNPS did. In vivo fluorescence imaging study on both subcutaneous KB mouse model and biologically relevant hamster cheek carcinogenesis further demonstrated rapid and enhanced accumulation/activation of eNPS over PS in tumor. Therefore, NPS platform demonstrate the potential to overcome the limitations of poor intracellular accumulation of porphysome to advance porphysome for multimodal cancer imaging and effective PDT.

In an aspect, there is provided a bilayer nanovesicle comprising porphyrin-phospholipid conjugate and a chelator-fatty acid conjugate; wherein the chelator-fatty acid conjugate comprises an aminopolycarboxylic acid conjugated to a single chain fatty acid; and the porphyrin-phospholipid conjugate comprises one porphyrin, porphyrin derivative or porphyrin analog covalently attached to a lipid side chain, preferably at the sn-1 or the sn-2 position, of one phospholipid.

In some embodiments, the aminopolycarboxylic acid is glycinate, IDA, NTA, EDTA, DTPA, EGTA, BAPTA, NOTA, DOTA, Nicotianamine, EDDHA, or EDDS. Preferably, the aminopolycarboxylic acid is EDTA or DTPA.

In some embodiments, the single chain fatty acid comprises 10 to 26 carbons.

In some embodiments, the single chain fatty acid comprises 12 to 22 carbons. Preferably, the single chain fatty acid comprises 14 to 18 carbons. Further preferably, the single chain fatty acid comprises 16 carbons. In an embodiment, the single chain fatty acid is hexadecylamide.

In some embodiments, the chelator-fatty acid conjugate is EDTA-hexadecylamide or DTPA-hexadecylamide.

In some embodiments, the bilayer nanovesicle comprises between 15%-60% chelator-fatty acid conjugate, preferably, between 25%-50% chelator-fatty acid conjugate, further preferably between 30-40% chelator-fatty acid conjugate, further preferably, about 30% chelator-fatty acid conjugate.

In some embodiments, the bilayer nanovesicle comprises between 1-60 molar % porphyrin-phospholipid conjugate, preferably between 20-40 molar % porphyrin-phospholipid conjugate, further preferably, about 27 molar % porphyrin-phospholipid conjugate.

In some embodiments, the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

Preferably, the expanded porphyrin is a texaphyrin, a sapphyrin or a hexaphyrin and the porphyrin isomer is a porphycene, an inverted porphyrin, a phthalocyanine, or a naphthalocyanine.

In some embodiments, the phospholipid in the porphyrin-phospholipid conjugate comprises phosphatidylcholine, phosphatidylethanoloamine, phosphatidylserine or phosphatidylinositol. Preferably, the phospholipid comprises an acyl side chain of 12 to 22 carbons.

In some embodiments, the porphyrin in the porphyrin-phospholipid conjugate is pyropheophorbide-a acid.

In some embodiments, the porphyrin in the porphyrin-phospholipid conjugate is a bacteriochlorophyll derivate.

In some embodiments, the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

In some embodiments, the porphyrin-phospholipid conjugate is pyro-lipid.

In some embodiments, the porphyrin-phospholipid conjugate is oxy-bacteriochlorophyll-lipid, texaphyrin-phospholipid conjugate or Aza-boron dipyrromethene (BODIPY)-phospholipid conjugate.

In some embodiments, the porphyrin is conjugated to the glycerol group on the phospholipid by a carbon chain linker of 0 to 20 carbons.

In some embodiments, the bilayer nanovesicle further comprises a PEGylated emulsifier. Preferably, the PEGylated emulsifier has a molecular weight ranging from about 1000 to about 5000. Further preferably, the PEGylated emulsifier is selected from the group consisting of N-(methoxypolyethylene glycol 5000 carbamoyl)-1,2-dipalmitoyl-sn-glycero-3-phosphatidylethanolamine (MPEG5000-DPPE), 1,2-dimyristoyl-sn-glycero-3-phosphoethanolamine-N-[methoxy(polyethylene glycol)-2000 (DMPE-PEG2000), 1,2-distearoyl-sn-glycero-3-phosphoethanolamine-N-[methoxy(polyethylene glycol)-2000 (DSPE-PEG2000), Polyoxyethylene 40 stearate (PEG40S) and combinations thereof.

In some embodiments, the PEG or PEG-lipid is present in an amount between 1-10 molar %. Preferably, the PEG or PEG-lipid is present in an amount between 2-7 molar %.

In some embodiments, the bilayer nanovesicle further comprises cholesterol.

In some embodiments, the remaining composition of the bilayer nanovesicle substantially comprises the cholesterol.

In some embodiments, the cholesterol is present in an amount between 1-60 molar %.

In some embodiments, the bilayer nanovesicle is substantially spherical.

In some embodiments, the bilayer nanovesicle is between about 70-120 nm in diameter. Preferably, the bilayer nanovesicle is between about 90-100 nm in diameter.

In some embodiments, the porphyrin-phospholipid conjugate comprises a metal chelated therein, optionally a radioisotope of a metal.

In an aspect, there is provided a composition comprising the bilayer nanovesicles described herein in a buffer.

In an aspect, there is provided a method of performing photodynamic therapy to a target area on a subject, comprising: providing the composition described herein; administering the composition to the subject; and irradiating the target area with light of a wavelength that excites the composition to create radicals and/or reactive oxygen species.

In an aspect, there is provided a method of imaging a target area in a subject, comprising providing the composition described herein; administering the composition to the subject; and measuring and/or detecting fluorescence or photoacoustic signal at the target area.

In an aspect, there is provided a method of delivering a radioisotope to a subject comprising: providing the composition described herein, wherein the bilayer nanovesicle has a radioisotope chelated therein; and administering the composition to the subject.

In an aspect, there is provided a use of the composition described herein for performing photodynamic therapy.

In an aspect, there is provided a use of the composition described herein for performing imaging.

In an aspect, there is provided a use of the composition described herein for delivering a radioisotope to a subject.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLES

### Methods and Materials

### Materials and Reagents

Cholesterol, 1,2-distearoyl-sn-glycero-3-phosphoethanolamine-N-[amino (polyethylene glycol)-2000] (DSPE-PEG 2000), 1,2-dipalmitoyl-sn-glycero-3-phosphoethanolamine-N-diethylenetriaminepentaacetic acid (16:0 PE-DTPA), 1,2-dipalmitoyl-sn-glycero-3-phosphoethanolamine-N-diethylenetriaminepentaacetic acid (gadolinium salt) (16:0 PE-DTPA(Gd)) and diethylenetriaminepentaa cetic acid)-bis(stearylamide) (gadolinium salt) (DTPA-BSA(Gd)) were purchased from Avanti Polar Lipids (USA). All other solvents and reagents were obtained from Sigma Aldrich. Porphyrin-lipid was synthesized accordingly to a previously published method¹¹. EDTA-hexadecylamide conjugate (EDTA-lipid) and DTPA-hexadecylamide conjugate (DTPA-lipid) were synthesized (see supporting information). Hoechst 33258 and LIVE/DEAD® Viability/Cytotoxicity kits were purchased from Invitrogen Corporation (Carlsbad, CA, USA). Mitotracker Green FM or Lysotracker Red DND-99 were obtained from Invirtogen/Thermo Fisher

Chromatographic purifications were performed using flash chromatography (Biotage, Isolera™). The UPLC-MS was performed using a Waters Acuity UPLC© Peptide BEH C18 column (130 Å, 1.7 μm, 2.1 mm×50 mm) with a Waters 2695 controller, a 2996 photodiode array detector, and a Waters triple quadrupole (TQ) mass detector (Waters Canada, Ontario, Canada). The UPLC conditions were as follows: Solvent A) 0.1% TFA and B) acetonitrile; column temperature: 60° C.; flow rate: 0.6 mL min-1; gradient from 80% A+20% B to 0% A+100% B in 5 min, kept at 100% B for 2 min, followed by a sharp change back to 80% A+20% B for 1 min. NMR spectra were recorded on a Bruker Ultrashield 400 Plus NMR spectrometer that measure 1D 1H NMR, 2D COSY 1H NMR 400.18 MHz. All measurements were referenced against and internal standard tetramethylsilane (TMS).

### EDTA Monoanhydride Synthesis

Ethylenediaminetetraacetic dianhydride (3.2 g, 0.0125 mol) was added followed by addition of dry DMF (20 mL) under Ar and heated to 80° C. After 15 minutes of stirring, water (0.225 mL) was added dropwise and reaction mixture allowed to reflux for 1 h 45 min under Ar. The crude was filtered and washed with DMF and ether to yield a white solid (1.95 g, 61%). The molecular structure were identified by ¹H NMR (400 MHz, DMSO-d6) δ 12.32 (s, 2H, CO2H), 3.72 (s, 4H), 3.43 (s, 4H), 2.83-2.74 (m, 2H), 2.58 (t, J=6.1 Hz, 2H).

### EDTA-Hexadecylamide Lipid (EDTA-Lipid)

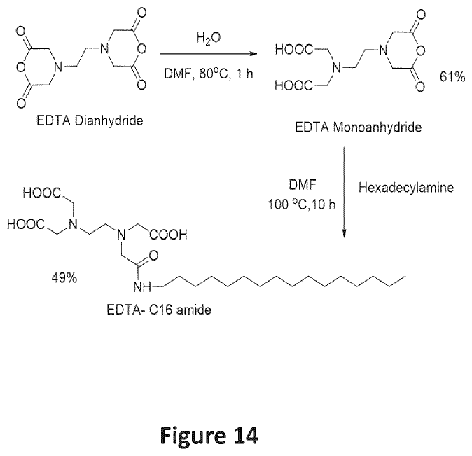

EDTA monoanhydride (1.42 g, 5.2 mmol) and hexadecylamine (1.2 g, 5 mmol) was dissolved in dry DMF (60 mL) and refluxed at 100° C. under Ar for 10 h. The reaction mixture was cooled to room temperature and poured into water to precipitate and filtrated. The crude was wash with water and diethyl ether to yield a white solid (1.25 g, 49%). The molecular structure were confirmed by ¹H NMR (400 MHZ, DMSO-d6) δ 8.01 (s, 1H, NH), 3.42 (s, 4 H), 3.36 (s, 2H), 3.18 (s, 2H), 3.05 (d, J=7.0 Hz, 2H), 2.79-2.63 (m, 4H), 1.38 (s, 2H), 1.30-1.14 (m, 26H), 0.85 (t, J=6.6 Hz, 3H). UPLC-MS (ESI): 516 m/z [M+H]+.tr=1.3 min. See FIG. 14.

### NPS Synthesis and Characterization

NPS were synthesized following previously reported protocol for porphysome formulation¹¹. The lipid components consists of porphyrin-lipid (pyropheophorbide-lipid), cholesterol (Avanti Polar Lipids, Alabaster, AL), distearoyl-sn-glycero-3-phosphoethanolamine-N-methoxy(polyetheneglycol) (PEG2000-DSPE, Avanti Polar Lipids), and EDTA/DTPA lipids at different molar ratio were well mixed and dissolved in chloroform. The Lipid mixtures were dried under a gentle stream of nitrogen gas and additional 1 h vacuuming. The dried lipid films were then rehydrated with PBS buffer (150 mM, pH 7.5) at the concentration of 3 mg/mL, undergone freeze-and thaw process for 9 times, and extruded through a polycarbonate membrane (pore size=100 nm) for 10 times. To determine the morphology of NPS, the particles were stained with 2% uranyl acetate negative staining, and then scanned with a Hitachi H-7000 electron microscope (Hitachi High Technologies America, Inc. Illinois, USA). The size and distribution were measured by dynamic light scattering (ZS90 Nanosizer, Malvern Instruments). In the spectroscopic studies, PLP was diluted either in PBS as intact/quenched sample or in PBS containing 0.5% Triton X-100 as disrupted/unquenched sample. The absorption and fluorescence spectra of the intact and disrupted NPS were measured respectively, by UV/Vis spectrophotometer Cary 50 (Agilent, Mississauga, ON) and Fluoromax-4 fluorometer (Horiba Jobin Yvon, USA) (Excitation: 420 nm, Emission: 630-800 nm, slit width: 5 nm). The porphyrin fluorescence quenching efficiency was calculated using the following equation.

$$Quenching efficiency = ( 1 - {F}_{i} / {F}_{d} ) \times 100 \%$$

Fᵢ and F_{d} stand for the fluorescence intensity of the intact and the corresponding disrupted NPS and the porphyrin concentration of 1 μM.

### Intracellular Uptake Assessment

Fluorescence microscopy method was firstly used to track particles' intracellular uptake on KB cells that cultured in RPMI-1640 media with 10% FBS. Briefly, 5×10⁴ cells/well were seeded in eight-well chamber slides 24 h prior to incubation. Cells were incubated with NPS and PS at porphyrin concentration of 1 μM for 4 h at 37° C., rinsed with PBS for 3 times and then re-culture in fresh media. Olympus FV1000 laser fluorescence scanning microscopy (Olympus, Tokyo, Japan) was conducted to monitor the porphyrin fluorescence change of the cells with time (immediately and at 3 h and 18 h after removing the incubation medium). The fluorescence microscopy method was also used to examine eNPS uptake in KB epithelium cells versus normal fibroblast cells (NFB)

To further compare the cellular uptake of NPS versus PS, a quantitative cellular uptake study was performed. KB cells were seeded in 12-well plate at 10⁶ cells per well 24 h prior to incubation. Cells were then incubated with various NPS and PS at varied porphyrin concentration for different time duration at 37° C. Following 3 times rinse with PBS, the cells were trypsinized and the suspension was centrifuged at 4,000 rpm for 5 min. The cell pellets containing 2.5×10⁵ cells were then re-suspended in 500 μL lysis buffer (DMSO) and incubated for 1 h. The solution was centrifuged at 10,000 rpm for 10 min and the supernatants were collected for porphyrin fluorescence measurement using Fluoromax-4 fluorometer to quantify the uptake of porphyrin molecule in cells.

### Serum Stability Assessment

The serum stability of various eNPS, LC-eNPS and PS (1 μM) was conducted by incubation with 50 vol % of FBS in PBS at 37° C. for different time duration and determining the fluorescence quenching efficiency changes of samples using a CLARIOstar microplate plate reader (BMG LABTECH) (excitation: 410/8 nm, emission: 671/8 nm, gain=2500).

### Evaluation of Intracellular Uptake Pathway of NPS

The uptake of eNPS and dNPS in KB cells at different temperature was evaluated using fluorescence microscopy. After 3 h incubation with eNPS and dNPS at 4, 18, and 37° C., cells were imaged and fluorescence signal at each temperature condition was compared to determine if eNPS and dNPS were actively or passively taken up by cancer cells. Their uptake was further evaluated under an adenosine-triphosphate (ATP)-depleted cell conditions by using sodium azide (NaN₃) and 2-deoxy-D-glucose (2-DG). KB cells were pre-incubated with NaN₃ and 2-DG 30 minutes prior to one hour of co-incubation with eNPS and dNPS, followed by fluorescence microscopy. Changes in fluorescence between normal and ATP-depleted cell conditions were assessed.

The subcellular localization of eNPS and dNPS was investigated on KB cells under confocal microscopy. The subcellular localization of eNPS and dNPS in mitochondria and lysosome were evaluated by co-localization assessment with mitochondria tracker, Mitotracker Green FM and lysosome tracker, Lysotracker Red DND-99. KB cells were incubated with nanoparticles for 24 h prior to incubation with the organelle trackers. The concentration and incubation time used for these trackers were optimized following manufacturer's recommendation. Confocal imaging setting: Porphyrin channel: excitation: 633 nm/emission: 670-740nm; Lysotracker channel: exitation: 552/emission: 573-620 nm, and mitotracker channel (excitation:488 nm/emission:510-600 nm).

### In Vitro PDT Assay

In vitro PDT activation of eNPS, LC-eNPS, and PS was determined by measuring KB cell viability after treatment. KB cells were incubated with 5 μM eNPS, LC-eNPS, and PS for 3, 6, and 24 h prior to light irradiation (660 nm; irradiance: 30 mW/cm²; light dose: 5 and 10 J/cm²) using a 660 nm home-made LED light box. Cell viability was assessed at 24 h post light treatment using alamarblue assay, where cells are incubated with 50 μg/mL of alamarblue for 2 h prior to fluorescence measurement. Alamarblue fluorescence was then measured by exciting at 540 nm and collecting the emission at 590 nm using CLARIOstar microplate reader. The cells cultured in regular medium was used as no treated control. Cells treated with porphysome but cultured in dark were used to evaluate the dark toxicity of each formulation. The cell viability of all samples was normalized to no treated control.

### In Vivo Fluorescence Imaging

All animals received humane care in accordance with the policies formulated by the University Health Network (UHN) Animal Care Committee, the Animal for Research Act of the Province of Ontario, and the Canadian Council on Animal Care. All animal studies have been approved by the UHN Animal Care Committee protocols. A KB subcutaneous mouse model and a hamster cheek carcinogenesis model were used throughout the study.

To develop KB xenograft model, athymic female nude mice at age of 8 weeks were inoculated subcutaneously with 2×10⁶ KB in 200 μL of PBS media on the right flank under general anesthesia (isofluorane in oxygen). Animals were maintained in pathogen-free conditions in autoclaved microisolator cages in the UHN Animal Resource Centre. When tumor size reached averaged size of 4.0 to 5.0 mm in diameter, mice were randomly categorized (n=3 for each group) and intravenously injected with PS and eNPS at a dose of 4 mg/kg of porphyrin. In vivo whole body imaging was conducted at different time points using Maestro imaging system ((CRI, USA) with a 575-605 nm excitation/645 nm long-pass emission filters to compare the tumor accumulation and activation while the mice were anesthetized by 2% (v/v) isoflurane. A low-fluorescence diet (Harlan Tekland®, Product No. TD.97184) were given to animal 3 days before NPS administration.

For hamster studies, 6-8 weeks old male Syrian hamsters (Harlan, Indianapolis, USA) were used to develop a model to mimic the clinical manifestations of oral carcinoma in humans²⁶. Briefly, 0.5% DMBA (7,12-dimethylbenz(a)anthracene) in DMSO was applied with a non-absorbent material to both cheeks while the animals were anesthetized with isofluorane. Prior to the application, KimWipes were packed in the oral cavity to minimize spilling down the throat and were removed after the application was completed. This procedure was repeated three times a week, for a period of 16-20 weeks. Generally, visible tumors were observed after 10 weeks of DMBA application and may reach up to 5-10 mm in size. The PS and eNPS were administrated into hamsters via cephalic vein at a dose of 4 mg/kg of porphyrin concentration and subjected to a pre-clinical fluorescence endoscope (PINPOINT imaging system, Novadaq Technologies Inc., Mississauga, ON, Canada) with a laser excitation wavelength of 665 nm for in vivo fluorescence imaging and real-time videos were recorded for fluorescence intensity analysis.

### Statistical Analysis

A two-way ANOVA with Bonferroni correction were used to determine the statistical significance between experimental groups. P-values<0.05 were considered significant

### RESULTS AND DISCUSSION

### EDTA-Lipid-Based New Porphysome (eNPS) Design and Synthesis

To incorporate EDTA moiety into porphysome nanostructure, an amphiphilic EDTA-lipid has been designed and synthesized by conjugating EDTA monoanhydride with hexadecylamine to gain EDTA-mono C16 lipid conjugation (EDTA-lipid) (FIG. 14). The chemical structure was identified by NMR and uPLC-MS (See the synthesis and characterization in the supporting information).

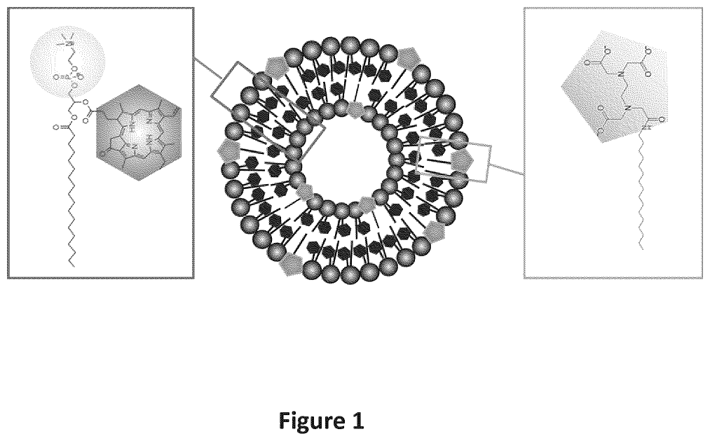

Various eNPSs were then made by varying EDTA-lipid proportion (18 mol %-50mol %) in porphysome formulation. The scheme of eNPS was illustrated in FIG. 1 and the components' contents for each formulation were listed in Table 1. The abbreviation of PS standards for the conventional porphysome formulation.

TABLE 1
Porphysome formulation components

| Porphysomes | PS | eNPS 1 | eNPS 2 | eNPS 3 |
|---|---|---|---|---|
| Cholesterol (mol %) | 40 | 43 | 29 | 17 |
| DSPE-PEG2000 (mol %) | 5 | 5 | 4 | 2 |
| Pyro-lipid (mol %) | 55 | 34 | 34 | 33 |
| EDTA-lipid (mol %) | 0 | 18 | 33 | 49 |

### eNPS Exhibited Structural and Photoproperty Similarity to Porphysome

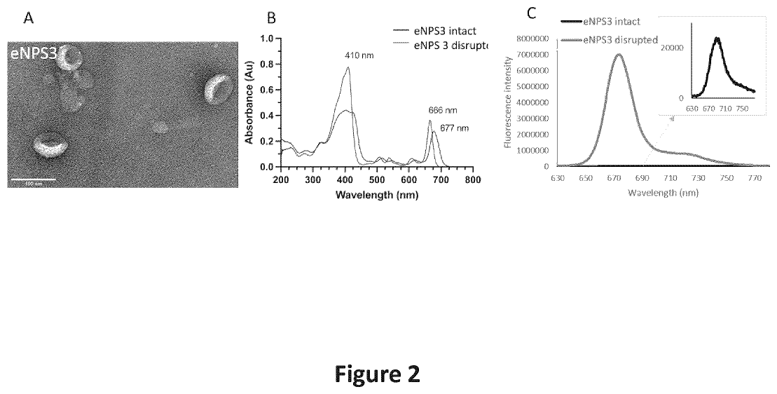

Incorporation of the EDTA-lipid (from 18-50 mol %) into porphysome formulation gave negligible effects in particles morphology (a liposome-like nanovesicle structure showed in TEM image of FIG. 2A), particle size (Z-average: 90-100 nm) and monodispersity (PDI: 0.14-0.18) (Table 2), indicating the compatibility of EDTA-lipid in porphysome formulation. In addition, eNPSs showed similar absorption spectra (FIG. 2B) and highly fluorescence self-quenching (over 150-fold) as PS did (FIG. 2C and Table 2), indicating minimal influence on intact porphysome' photoproperties.

TABLE 2
Porphysome characterization

| | | | | Fluorescence |
|---|---|---|---|---|
| | Zeta | Diameter | | quenching |
| Porphysomes | Potential (mV) | (nm) | PDI | fold |
| PS | −3.15 ± 0.53 | 96.2 ± 0.63 | 0.14 ± 0.02 | 179 |
| eNPS1 | −1.78 ± 0.13 | 93.7 ± 0.82 | 0.15 ± 0.02 | 164 |
| eNPS2 | −1.29 ± 0.10 | 90.5 ± 0.53 | 0.16 ± 0.03 | 202 |
| eNPS3 | −21.91 ± 0.45 | 97.4 ± 0.9 | 0.18 ± 0.01 | 272 |

PDI: Polydispersity index

### eNPS Showed Rapid and Enhanced Intracellular Uptake in KB Cells

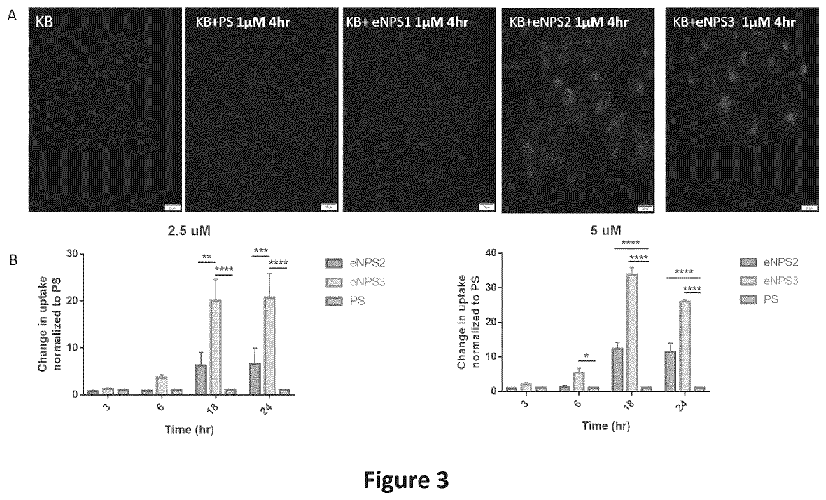

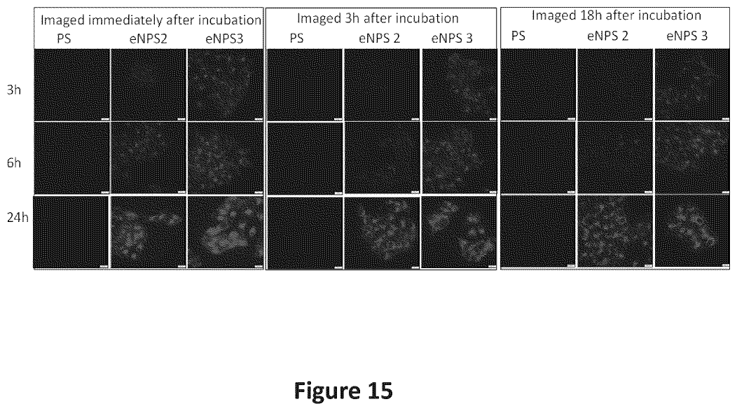

The intracellular uptake of various eNPS and PS was monitored by fluorescence microscopy, where KB cells were incubated with 1 μM of the particles for 4 h prior to imaging. As shown in FIG. 3A, eNPS2 and eNPS3 that contain over 30 mol % of EDTA-lipid exhibited rapid uptake and significantly enhanced fluorescence in KB cells when compared to PS. As fluorescence is highly quenched in intact eNPS, to monitor if it is time-consuming process for eNPS dissociation/activation after internalization, cells were culture continually in fresh medium after incubation. As shown in FIG. 15, fluorescence signal of all cells after incubation with eNPS2 and eNPS3 did not appear signal enhancement upon post-incubation while showed slowly fluorescence diminishing with increasing post-incubation time (3 h, 18 h), which suggests that eNPS undergo rapid activation within the intracellular environment.

Although eNPS showed rapid activation in cells, to quantify eNPS enhanced uptake over PS, a quantitative cell extraction method was further applied to determine time-dependent intracellular uptake profile of various particles. Using this method, the internalized eNPS and PS were completely disrupted in DMSO lysis solution, which unquenched porphyrin fluorescence for uptake quantification. As shown in FIG. 3B, a clear trend of enhanced uptake by increasing EDTA-lipid proportion in nanoparticle was observed. The eNPS3 with highest EDTA-lipid contents (50 mol %) demonstrated the highest uptake at all time points (p<0.05). A 2.1-, 5.5-, 33.7-, 26.1-fold enhanced uptake at 5 μM concentration compared to PS were observed for 3, 6, 18 and 24 h incubation, respectively. The eNPS2 contained 30 mol % of EDTA-lipid also showed significant enhanced delivery at a longer period of incubation, such as 12.3-and 11.3-fold enhanced uptake after 18 and 24 h incubation with 5 μM of particles, respectively (p<0.05). We choose eNPS3 as the optimal eNPS formulation for enhanced delivery evaluation in all subsequent studies.

### eNPS Triggered Selective Uptake in Epithelium Cells But Not in Fibroblast Cells

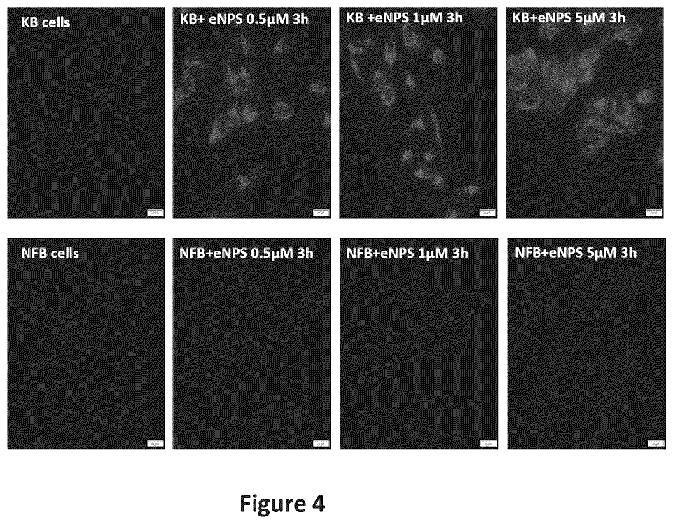

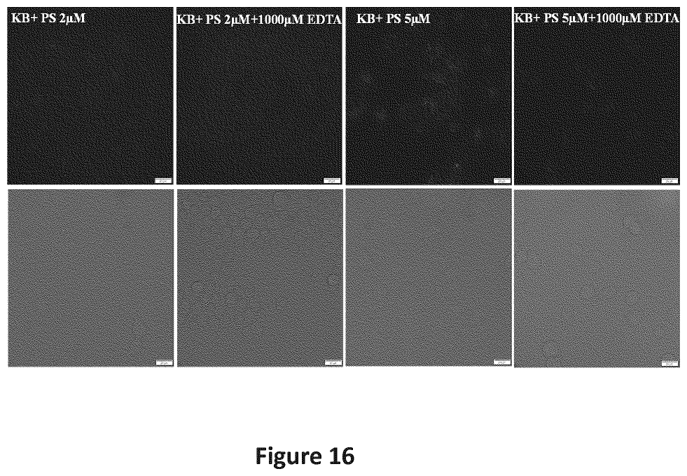

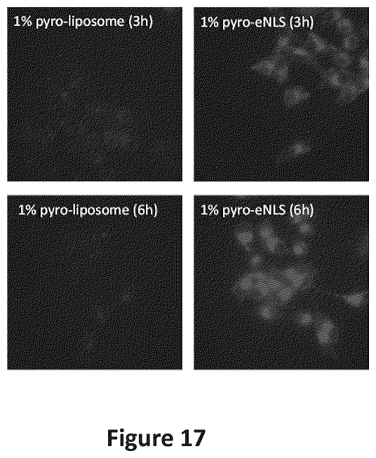

Fluorescence microscopy was used to assess the uptake of eNPS in epithelium cells KB vs. normal human fibroblast cells. Interestingly, it was found that eNPS significantly enhanced particles uptake in epithelium cells but not in fibroblast cells (FIG. 4). To investigate if eNPS-enhanced delivery in epithelium cells caused by EDTA heard group (5 μM of eNPS contains 7 μM of EDTA-lipid) interaction with divalent cations (e.g. Ca²⁺, Mg²⁺) that might increase cell membrane permeability, a high concentration of free EDTA (1000 μM) were added to co-incubate with regular PS (5 μM) to examine any effect on the PS uptake. The results (FIG. 16) revealed that 1 mM of free EDTA gave negligible enhancement on PS uptake. In addition, EDTA-lipid plays the same role on triggering liposome fast internalization in cells when incorporating EDTA-lipid into a liposome formulation (HSPC: EDTA-lipid: Pyro lipid: Cholesterol: DSPE-PEG2000=32: 49:1: 15: 2 (mol/mol)) (FIG. 17)

### DTPA-Lipid-Based New Porphysome (dNPS)

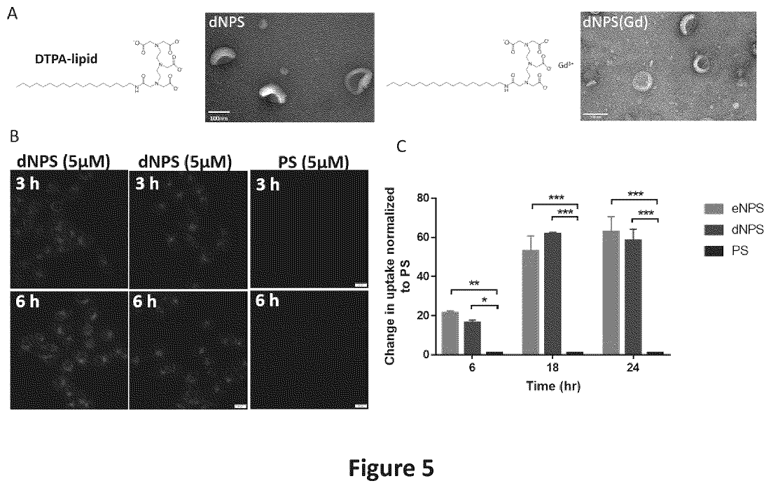

To investigate if NPS platform can be built up by replacing the tetraacetic acid head group of EDTA-lipid with other hydrophilic head groups, a diethylenetriaminepentaacetic acid-hexadecylamide (DTPA-lipid) was synthesised for NPS construction. The resultant dNPS (see in the formulation components detail in Table 3 and TEM images in FIG. 5A) showed similar enhanced delivery behavior as eNPS. As shown in FIG. 5B, dNPS and eNPS containing either 50 mol % of DTPA-lipid or EDTA-lipid showed similar enhanced fluorescence signal compared to PS at both 3 h and 6 h incubation time under fluorescence microscopy imaging, suggesting that the addition of a carboxylic acid group (5 groups in DTPA vs. 4 groups in EDTA) on the EDTA-lipid head group did not affect their uptake. A quantitative intracellular uptake study further confirmed non-significant difference between intracellular signal of dNPS and eNPS at each timepoint (6, 18, or 24 h) (p>0.05), suggesting that the cellular uptake profile of the two nanoparticles were similar (FIG. 5C). As well, both eNPS and dNPS had a significantly higher intracellular accumulation compared to PS at all time points (p<0.05). These data together suggest that both tetraactetic acid group of EDTA-lipid and pentaacetic acid head group of DTPA-lipid in the NPS platform are responsible for the drastically improved PS uptake.

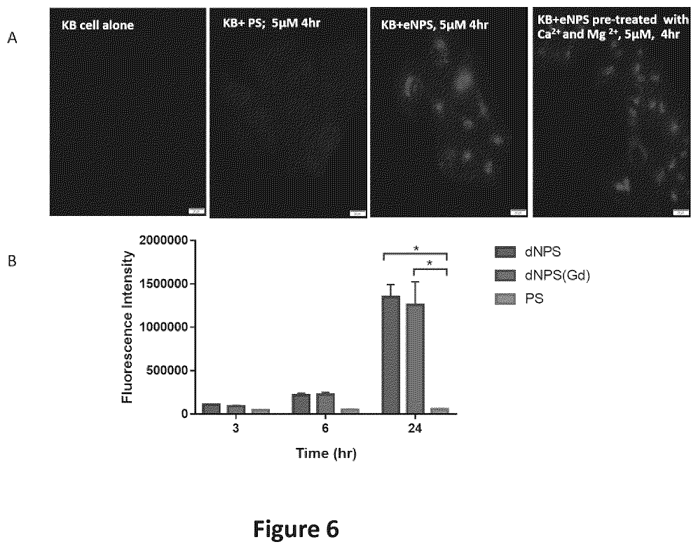

To investigate if EDTA chelation with Ca²⁺ or Mg²⁺ plays role in enhanced PS uptake, eNPS was pre-treated with high concentration of Ca²⁺ and Mg²⁺ before cell incubation. As shown in FIG. 6A, the Ca²⁺/Mg²⁺-treated eNPS showed similar enhanced uptake as untreated eNPS when compared with PS. In addition, the dNPS(Gd) made by Gd-chelated DTPA-lipid (FIG. 5A) showed similar intracellular uptake as metal-free dNPS (FIG. 6B) These results together with

TABLE 3
eNPS and dNPS formulation and characterization

| | Formulation Components | | | | | Particle characterization | |
|---|---|---|---|---|---|---|---|
| | EDTA/DTPA- | EDTA/DTPA- | Cholesterol | DSPE-PEG2000 | Pyro-lipid | Size ± SD | |
| Porphysome | lipid type | lipid (mol %) | (mol %) | (mol %) | (mol %) | (nm) | PDI ± SD |
| PS | — | 0 | 40 | 5 | 55 | 96.2 ± 0.6 | 0.14 ± 0.02 |
| eNPS | C16-EDTA | 48 | 17 | 2 | 33 | 97.4 ± 0.9 | 0.18 ± 0.01 |
| dNPS | C16-DTPA | 48 | 17 | 2 | 33 | 98.6 ± 2.1 | 0.30 ± 0.04 |
| dNPS(Gd) | C16-DTPA(Gd) | 48 | 17 | 2 | 33 | 103.5 ± 5.4 | 0.27 ± 0.02 |
| dNPS_{16PE} | 16:0 PE-DTPA | 48 | 17 | 2 | 33 | 51.0 ± 0.6 | 0.22 ± 0.01 |
| dNPS_{16PE}(Gd) | 16:0 PE-DTPA(Gd) | 48 | 17 | 2 | 33 | 90.3 ± 4.1 | 0.26 ± 0.01 |
| dNPS_{BSA}(Gd) | DTPA-BSA(Gd) | 48 | 17 | 2 | 33 | 123.0 ± 2.3 | 0.23 ± 0.03 |
| LC-eNPS | C16-EDTA | 38 | 30 | 5 | 27 | 84.4 ± 0.4 | 0.13 ± 0.01 |

Abbreviations: Standard deviation (SD);
polydispersity index (PDI)

the previous observation of no enhancement of PS uptake caused by 1 mM of free EDTA in FIG. 16, confirm that the mechanism of eNPS enhanced delivery is not dependent on EDTA chelation with Ca²⁺ or Mg²⁺, thus suggesting a previously unknown mechanism of action.

### Assess the Effects of Lipid Chain of Chelator-Lipid on NPS Uptake

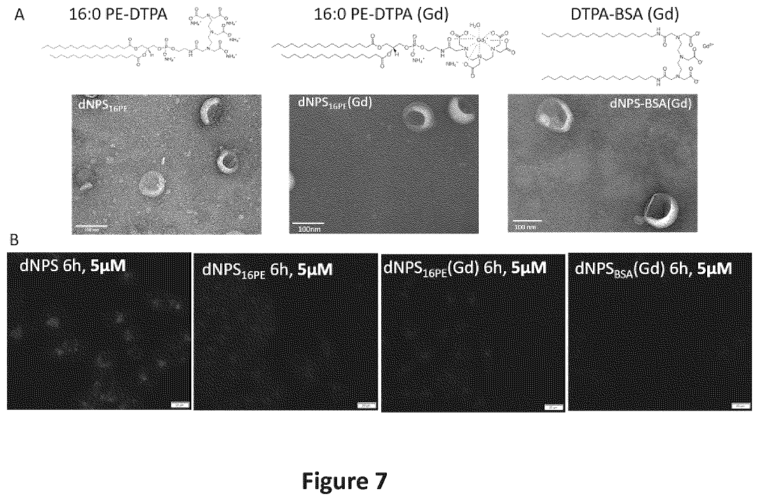

To investigate the effect of lipid chain of chelator-lipid on NPS uptake, DTPA-lipids with various double lipid chains including 16:0 PE-DTPA, 16:0 PE-DTPA(Gd) and DTPA-BSA(Gd) (their chemical structures shown in FIG. 7A) were incorporated into porphysome to form DPS_{16PE}, DPS_{16PE}(Gd) and DPS_{BSA}(Gd). The components and characterization of these formulations are summarized in Table 3 and their representative TEM images are shown in FIG. 7A. The eNPS and dNPS (containing a single fatty acid lipid chain) demonstrated significantly higher uptake compared to dNPS_{16PE}, dNPS_{16PE}(Gd) and dNPS_{BSA}(Gd) (containing double fatty acid lipid chains) variation (FIG. 7B), indicating that the single fatty acid lipid chain in eNPS or dNPS confers the highest uptake/unquenching of NPS.

### Characterizing the Effect of Cholesterol and PEG Content on Cell Uptake and Serum Stability of NPS

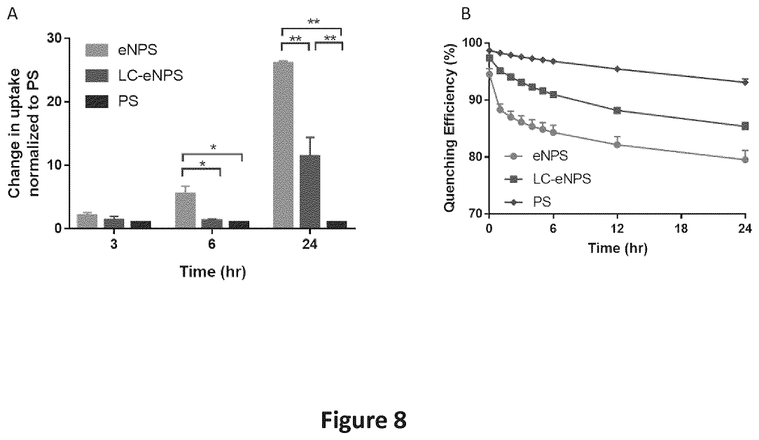

To further increase the serum stability and potential blood circulation time of NPS, a new eNPS formulation, termed LC-eNPS, was developed. LC-eNPS contains the same ratio of porphyrin-lipid: EDTA-lipid in NPS, but with increased cholesterol (30 mol %) and DSPE-PEG2000 (5 mol %) contents (Table 3). After 24 h incubation with 5 □M of nanoparticles, LC-eNPS maintained 10-fold enhanced intracellular uptake over PS but exhibited 50% less intracellular uptake when compared to eNPS (FIG. 8A). As fluorescence is highly quenched in intact porphysomes but unquenched when porphysome nanostructure is dissociated, the fluorescence quenching efficiency changing of NPSs was used to trace the particles' stability. After incubation with PBS containing 50 vol % of FBS, LC-eNPS appeared to have a higher stability compared to eNPS with higher fluorescence quenching at all time points and remained 85% quenched over a 24 h period (FIG. 8B), which suggests that increasing the cholesterol and PEG content to 30% and 5%, respectively, can improve the stability of eNPS in serum condition with a modest decrease in intracellular uptake efficiency.

### Investigate the Mechanism on Intracellular Uptake of NPS

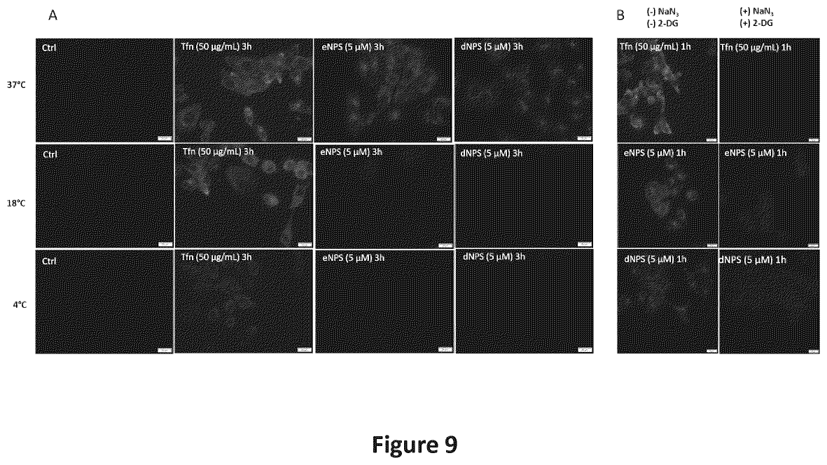

The general uptake mechanism (passive or active) of NPS in vitro was then investigated by temperature and energy modulation studies. Fluorescence microscopy was used to monitor their uptake. After KB cell incubation with eNPS and dNPS at different temperature conditions, a stronger fluorescence signal was observed at 37° C. compared to 18 and 4° C., which suggest that the rapid uptake of eNPS and dNPS was mostly via an active process (FIG. 9A). By creating ATP-depleted cell conditions using NaN₃ and 2-DG, it was observed that there was minimal fluorescence signal of eNPS and dNPS within cells compared to regular cultural conditions (FIG. 9B). This suggest that eNPS and dNPS are mainly actively transported into cells.

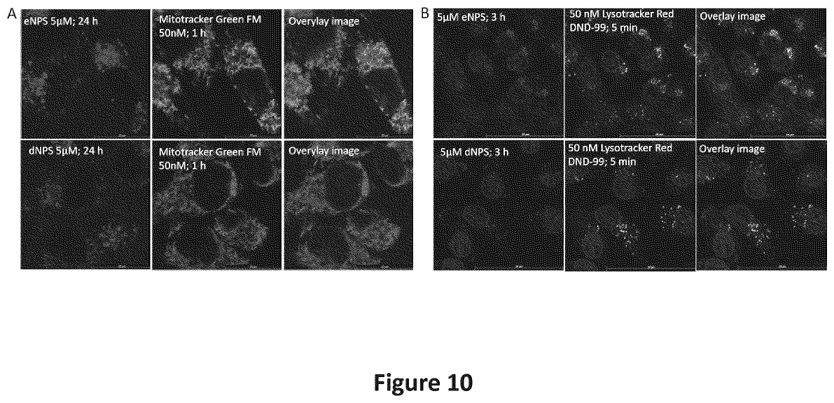

To gain insight of eNPS for PDT mechanism, subcellular localization of eNPS and dNPS was investigated on KB cells under confocal microscopy imaging. A punctate pattern of eNPS and dNPS signal was clearly observed, suggesting that both nanoparticles were not dispersed throughout the cytosol. By co-localization study with mitochondria and lysosome trackers, eNPS and dNPS did not appear to colocalize with either Mitotracker Green FM or Lysotracker Red DND-99 (FIG. 10). Further co-localization study with other organelle trackers, such as endoplasmic reticulum, golgi, and endosome trackers will be investigated.

### Evaluation of NPS for in Vitro PDT

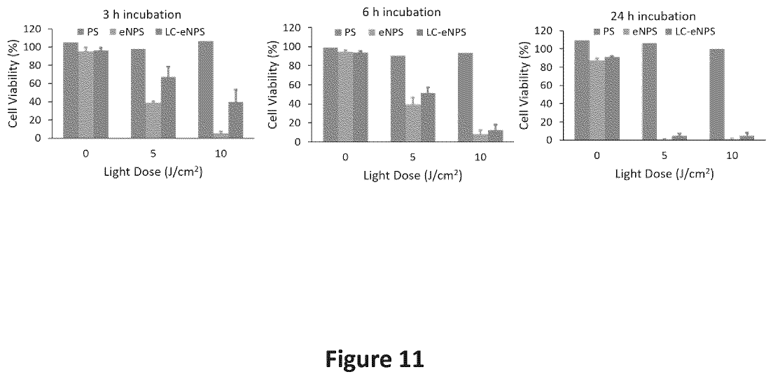

In vitro PDT activation of eNPS, LC-eNPS, and PS was evaluated in KB cells. The results showed in FIG. 11 demonstrated that 5 μM eNPS, PS, and LC-eNPS had minimal dark toxicity to cells after 24 h incubation. Upon light treatment, eNPS induced the largest reduction in cell viability at all timepoints when compared to other groups, suggesting that it had the highest PDT efficacy. LC-eNPS also caused greater reduction in cell viability and the resultant cell death was magnitudedly corresponding to LC-eNPS particles concentration and light doss. After 24 h cell incubation, both 5 μM eNPS and LC-eNPS caused>95% cell killing at light dose of 5 or 10 J/cm². However, PS treated cells showed minimal cell toxicity at all experimental condition (all concentration, incubation time, and light doses). The in vitro PDT potency trend of eNPS, LC-eNPS, and PS was well-correlated with their uptake efficiency (FIG. 8A). Therefore, enhanced delivery of eNPS and LC-eNPS triggered efficacious PDT eventually.

### Evaluate NPS Enhanced Delivery and Activation in Vivo

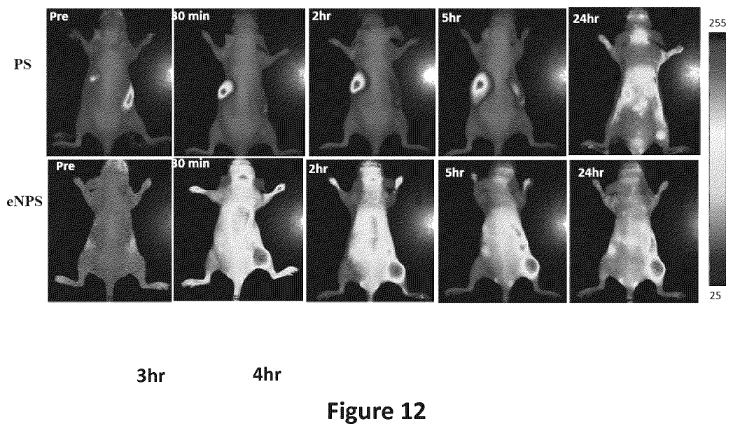

To assess the influence NPS on in vivo tumor accumulation and activation, equivalent doses of eNPS and PS (4 mg/kg) were intravenously administered to mice bearing KB subcutaneous tumor followed by whole-animal in vivo fluorescence imaging using a Maestro system (CRI, USA) over 24 h. The images were acquired using a 575-605 nm excitation filter and a 645 nm long-pass emission filter with exposure time of 200 ms. As shown in FIG. 12, eNPS demonstrated significant accumulation and activation in the tumor 30 min post injection and remained stable for 24 h (n=5), while PS showed negligible fluorescence signal in tumor within 5 h and much less signal at 24 h, indicating eNPS significantly enhanced porphysome accumulation and activation in tumor for successful tumor fluorescence imaging.

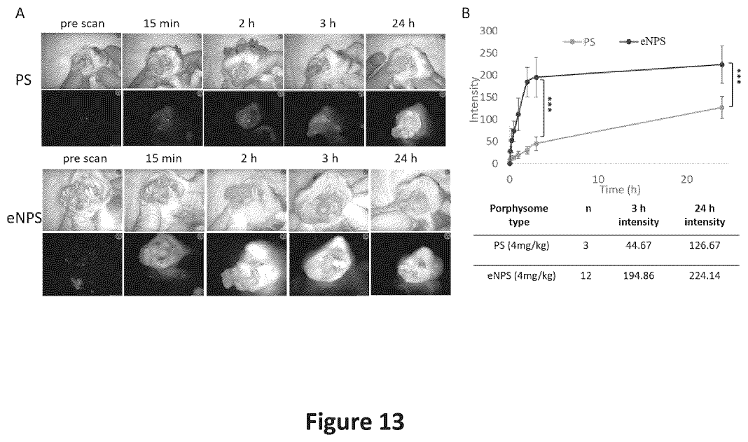

We then applied NPS-enhanced fluorescence imaging on a more biologically relevant hamster cheek carcinogenesis that produced by the repeated treatment of DMBA and closely mimics the clinical manifestations of oral carcinoma in humans²⁶. After intravenous injection of eNPS and PS, in vivo fluorescence imaging was conducted using a NOVADAQ Pinpoint system (Mississauga, Ontario) to visualize porphyrin fluorescence in hamster cheek tumor. As shown in FIG. 13A, tumor-specific strong fluorescence was detected at 15 min post injection of eNPS, peaked at 3 h and remain stable for 24 h, while PS group showed much weaker tumor signal at all time point. The tumor fluorescence intensity at different time points was quantified by the Pinpoint system and profiled in FIG. 13B, It was clear that enhanced fluorescence signal in tumor of eNPS group (n=12) vs. PS group (n=2) was observed through all time points, specially 4.4-fold enhanced signal at early time point of 3 h and 1.7-fold enhancement at 24 h. These data further demonstrated rapid and enhanced accumulation and activation of eNPS over PS in tumor. Therefore, NPS platform provide the potential to overcome the limitations of poor tumor accumulation of PS to advance porphysome for cancer imaging and effective PDT.

### In Vivo PDT

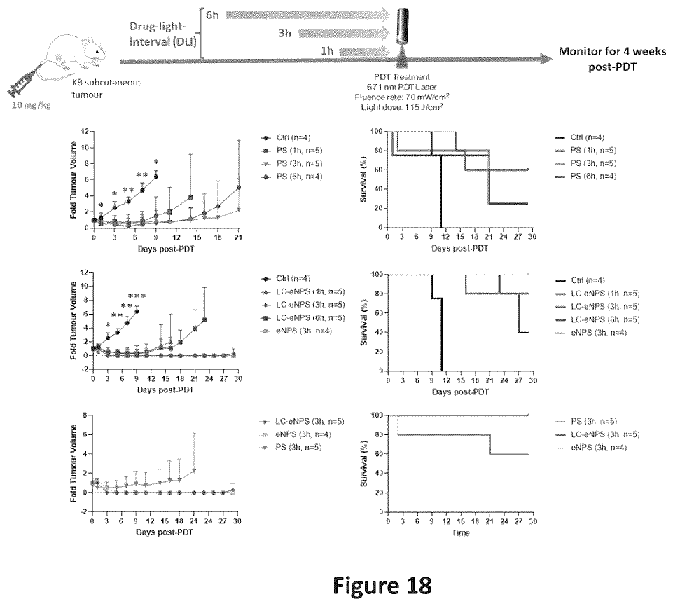

Referring to FIG. 18, after in vivo PDT pilot studies to determine the optimal laser treatment parameters, evaluation of in vivo PDT efficacy of eNPS, LC-eNPS, and PS at various drug-light-intervals was performed. Subcutaneous KB tumor bearing athymic nude mice were intravenously injected with eNPS, LC-eNPS or PS (10 mg/kg porphyrin concentration) followed by 671 nm laser irradiation (50 mW, 115 J/cm²) at 1-, 3-, and 6-hours post-injection. PDT efficacy was evaluated by monitoring mouse survival and changes in tumor size post-treatment for up to 4 weeks. The tumour sizes of all the PDT treatment groups were found to be statistically lower compared to that of the control group. Both eNPS and LC-eNPS (3 h DLI) demonstrated enhanced PDT efficacy compared to PS. The ePS and LC-ePS PDT treatment at 3 h DLI showed significant high rate of tumour complete ablation (100% and 80%, respectively) compared to 20% of PS at 3 h DLI.

### In Vivo Fluorescence Activation

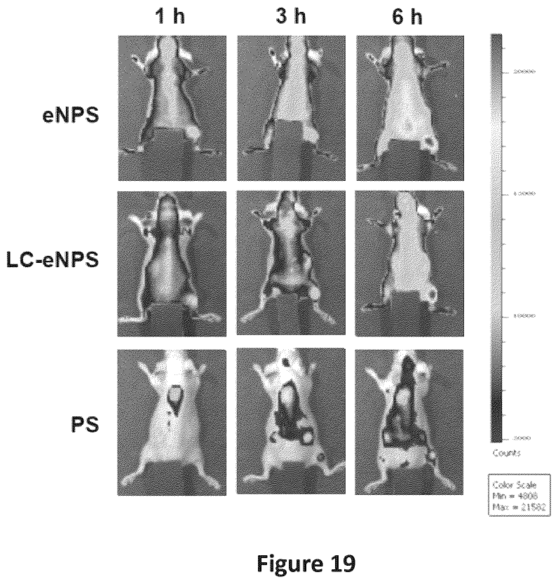

Referring to FIG. 19, to evaluate the extent of unquenched or “active” eNPS and LC-eNPS compared to PS available at the tumour site for PDT, the tumour fluorescence activation post-injection of these nanoparticles was monitored. Subcutaneous KB tumor bearing athymic nude mice were intravenously injected with eNPS, LC-eNPS or PS (10 mg/kg porphyrin concentration) followed by in vivo tumour fluorescence imaging at 1-, 3-, and 6-hours post-injection. Both eNPS and LC-eNPS demonstrated the highest in vivo fluorescence activation at all timepoints compared to PS.

In this study, the next generation porphysome platform (NPS) was developed by introducing single chain fatty acid EDTA-lipid in porphysome formulation, which demonstrated significantly enhanced intracellular tumor cell accumulation, resulting in efficacious PDT. The enhanced tumor accumulation and activation of NPS has been also validated in both subcutaneous mouse tumor model and biologically relevant hamster cheek carcinogenesis model. To our knowledge, this is the first report to incorporate EDTA-lipids into nanoparticle formulation to improve their biological and therapeutic properties. Importantly, the enhanced delivery of nanoparticles by EDTA-lipid is via a previously unknown mechanism and can be extended to the single fatty acid DTPA-lipid conjugates but not to other double fatty acid DTPA-lipids. Aside from their phototherapeutic potential, NPSs show great promise for fluorescence-guided surgery applications due to their preferential rapid uptake and activated fluorescence of guidance. The additional metal/radioisotope chelation abilities of EDTA-lipid/DTPA-lipids complement the intrinsic metal chelating properties of porphysomes, allowing NPSs to deliver a wide spectrum of radioisotopes for multimodal imaging and radiotherapy. Therefore, NPS platform demonstrate the potential to overcome the limitations of poor intracellular accumulation of porphysome to advance porphysome for multimodal cancer imaging and effective PDT.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Hopper, C. Photodynamic Therapy: A Clinical Reality in the Treatment of Cancer. The Lancet. Oncology 2000, 1, 212-219.
2. Nyst, H. J.; Tan, I. B.; Stewart, F. A.; Balm, A. J. Is Photodynamic Therapy a Good Alternative to Surgery and Radiotherapy in the Treatment of Head and Neck Cancer? Photodiagnosis and photodynamic therapy 2009, 6, 3-11.
3. Stewart, F. A. Re-Treatment after Full-Course Radiotherapy: Is It a Viable Option? Acta oncologica 1999, 38, 855-862.
4. Dabrowski, J. M.; Arnaut, L. G. Photodynamic Therapy (Pdt) of Cancer: From Local to Systemic Treatment. Photochemical & photobiological sciences: Official journal of the European Photochemistry Association and the European Society for Photobiology 2015, 14, 1765-1780.
5. Dolmans, D. E.; Fukumura, D.; Jain, R. K. Photodynamic Therapy for Cancer. Nature reviews. Cancer 2003, 3, 380-387.
6. Ethirajan, M.; Chen, Y.; Joshi, P.; Pandey, R. K. The Role of Porphyrin Chemistry in Tumor Imaging and Photodynamic Therapy. Chemical Society reviews 2011, 40, 340-362.
7. Huang, Z. A Review of Progress in Clinical Photodynamic Therapy. Technology in cancer research & treatment 2005, 4, 283-293.
8. Li, X.; Lovell, J. F.; Yoon, J.; Chen, X. Clinical Development and Potential of Photothermal and Photodynamic Therapies for Cancer. Nature reviews. Clinical oncology 2020, 17, 657-674.
9. Rajora, M. A.; Lou, J. W. H.; Zheng, G. Advancing Porphyrin's Biomedical Utility Via Supramolecular Chemistry. Chemical Society reviews 2017, 46, 6433-6469.
10. Otvagin, V. F.; Kuzmina, N. S.; Krylova, L. V.; Volovetsky, A. B.; Nyuchev, A. V.; Gavryushin, A. E.; Meshkov, I. N.; Gorbunova, Y. G.; Romanenko, Y. V.; Koifman, O. I.; Balalaeva, I. V.; Fedorov, A. Y. Water-Soluble Chlorin/Arylaminoquinazoline Conjugate for Photodynamic and Targeted Therapy. Journal of medicinal chemistry 2019, 62, 11182-11193.
11. Lovell, J. F.; Jin, C. S.; Huynh, E.; Jin, H.; Kim, C.; Rubinstein, J. L.; Chan, W. C.; Cao, W.; Wang, L. V.; Zheng, G. Porphysome Nanovesicles Generated by Porphyrin Bilayers for Use as Multimodal Biophotonic Contrast Agents. Nature materials 2011, 10, 324-332.
12. Lovell, J. F.; Jin, C. S.; Huynh, E.; MacDonald, T. D.; Cao, W.; Zheng, G. Enzymatic Regioselection for the Synthesis and Biodegradation of Porphysome Nanovesicles. Angewandte Chemie 2012, 51, 2429-2433.
13. Liu, T. W.; Macdonald, T. D.; Jin, C. S.; Gold, J. M.; Bristow, R. G.; Wilson, B. C.; Zheng, G. Inherently Multimodal Nanoparticle-Driven Tracking and Real-Time Delineation of Orthotopic Prostate Tumors and Micrometastases. ACS nano 2013, 7, 4221-4232.
14. Liu, T. W.; MacDonald, T. D.; Shi, J.; Wilson, B. C.; Zheng, G. Intrinsically Copper-64-Labeled Organic Nanoparticles as Radiotracers. Angewandte Chemie 2012, 51, 13128-13131.
15. MacDonald, T. D.; Liu, T. W.; Zheng, G. An Mri-Sensitive, Non-Photobleachable Porphysome Photothermal Agent. Angewandte Chemie 2014, 53, 6956-6959.
16. Jin, C. S.; Cui, L.; Wang, F.; Chen, J.; Zheng, G. Targeting-Triggered Porphysome Nanostructure Disruption for Activatable Photodynamic Therapy. Advanced healthcare materials 2014, 3, 1240-1249.
17. Chisolm, J. J., Jr. Bal, Edta, Dmsa and Dmps in the Treatment of Lead Poisoning in Children. Journal of toxicology. Clinical toxicology 1992, 30, 493-504.
18. Domingo, J. L. Developmental Toxicity of Metal Chelating Agents. Reproductive toxicology 1998, 12, 499-510.
19. Asbell, M. A.; Eagon, R. G. The Role of Multivalent Cations in the Organization and Structure of Bacterial Cell Walls. Biochemical and biophysical research communications 1966, 22, 664-671.
20. Yu, Q.; Wang, Z.; Li, P.; Yang, Q. The Effect of Various Absorption Enhancers on Tight Junction in the Human Intestinal Caco-2 Cell Line. Drug development and industrial pharmacy 2013, 39, 587-592.
21. Born, T.; Kontoghiorghe, C. N.; Spyrou, A.; Kolnagou, A.; Kontoghiorghes, G. J. Edta Chelation Reappraisal Following New Clinical Trials and Regular Use in Millions of Patients: Review of Preliminary Findings and Risk/Benefit Assessment. Toxicology mechanisms and methods 2013, 23, 11-17.
22. Duvillard, C.; Ponelle, T.; Chapusot, C.; Piard, F.; Romanet, P.; Chauffert, B. Edta Enhances the Antitumor Efficacy of Intratumoral Cisplatin in S.C. Grafted Rat Colon Tumors. Anti-cancer drugs 2004, 15, 295-299.
23. Elihu, N.; Anandasbapathy, S.; Frishman, W. H. Chelation Therapy in Cardiovascular Disease: Ethylenediaminetetraacetic Acid, Deferoxamine, and Dexrazoxane. Journal of clinical pharmacology 1998, 38, 101-105.
24. Fulgenzi, A.; Ferrero, M. E. Edta Chelation Therapy for the Treatment of Neurotoxicity. International journal of molecular sciences 2019, 20.
25. Song, Y.; Huang, Z.; Song, Y.; Tian, Q.; Liu, X.; She, Z.; Jiao, J.; Lu, E.; Deng, Y. The Application of Edta in Drug Delivery Systems: Doxorubicin Liposomes Loaded Via Nh4edta Gradient. International journal of nanomedicine 2014, 9, 3611-3621.
26. Chen, Y. K.; Lin, L. M. Dmba-Induced Hamster Buccal Pouch Carcinoma and Vx2-Induced Rabbit Cancer as a Model for Human Oral Carcinogenesis. Expert review of anticancer therapy 2010, 10, 1485-1496.

1. A bilayer nanovesicle comprising porphyrin-phospholipid conjugate and a chelator-fatty acid conjugate; wherein
the chelator-fatty acid conjugate comprises an aminopolycarboxylic acid conjugated to a single chain fatty acid; and
the porphyrin-phospholipid conjugate comprises one porphyrin, porphyrin derivative or porphyrin analog covalently attached to a lipid side chain, preferably at the sn-1 or the sn-2 position, of one phospholipid.

2. The bilayer nanovesicle of claim 1, wherein the aminopolycarboxylic acid is glycinate, IDA, NTA, EDTA, DTPA, EGTA, BAPTA, NOTA, DOTA, Nicotianamine, EDDHA, or EDDS.

3. The bilayer nanovesicle of claim 2, wherein the aminopolycarboxylic acid is EDTA or DTPA.

4. The bilayer nanovesicle of claim 1, wherein the single chain fatty acid comprises 10 to 26 carbons.

5. The bilayer nanovesicle of claim 4, wherein the single chain fatty acid comprises 12 to 22 carbons, 14 to 18 carbons, or 16 carbons.

6. (canceled)

7. (canceled)

8. The bilayer nanovesicle of claim 5, wherein the single chain fatty acid is hexadecylamide.

9. The bilayer nanovesicle of claim 1, wherein the chelator-fatty acid conjugate is EDTA-hexadecylamide or DPTA-hexadecylamide.

10. The bilayer nanovesicle of claim 1, comprising between 15%-60% chelator-fatty acid conjugate, between 25%-50% chelator-fatty acid conjugate, between 30-40% chelator-fatty acid conjugate, or about 30% chelator-fatty acid conjugate.

11. (canceled)

12. (canceled)

13. (canceled)

14. The bilayer nanovesicle of claim 1, comprising between 1-60 molar % porphyrin-phospholipid conjugate.

15. The bilayer nanovesicle of claim 14 comprising between 20-40 molar % porphyrin-phospholipid conjugate or about 27 molar % porphyrin-phospholipid conjugate.

16. (canceled)

17. The bilayer nanovesicle of claim 1, wherein the porphyrin, porphyrin derivative or porphyrin analog in the porphyrin-phospholipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

18. The bilayer nanovesicle of claim 17, wherein the expanded porphyrin is a texaphyrin, a sapphyrin or a hexaphyrin and the porphyrin isomer is a porphycene, an inverted porphyrin, a phthalocyanine, or a naphthalocyanine.

19. The bilayer nanovesicle of claim 1, wherein the phospholipid in the porphyrin-phospholipid conjugate comprises phosphatidylcholine, phosphatidylethanoloamine, phosphatidylserine or phosphatidylinositol.

20. The bilayer nanovesicle of claim 19, wherein the phospholipid comprises an acyl side chain of 12 to 22 carbons.

21. The bilayer nanovesicle of claim 1 wherein the porphyrin in the porphyrin-phospholipid conjugate is pyropheophorbide-a acid or a bacteriochlorophyll derivate.

22. (canceled)

23. The bilayer nanovesicle of claim 1 wherein the phospholipid in the porphyrin-phospholipid conjugate is 1-Palmitoyl-2-Hydroxy-sn-Glycero-3-Phosphocholine or 1-Stearoyl-2-Hydroxy-sn-Gycero-3-Phosphocholine.

24. The bilayer nanovesicle of claim 1 wherein the porphyrin-phospholipid conjugate is pyro-lipid.

25. The bilayer nanovesicle of claim 1 wherein the porphyrin-phospholipid conjugate is oxy-bacteriochlorophyll-lipid, texaphyrin-phospholipid conjugate or Aza-boron dipyrromethene (BODIPY)-phospholipid conjugate.

26. The bilayer nanovesicle of claim 1 wherein the porphyrin is conjugated to the glycerol group on the phospholipid by a carbon chain linker of 0 to 20 carbons.

27. The bilayer nanovesicle of claim 1 further comprising a PEGylated emulsifier.

28. The bilayer nanovesicle of claim 27 wherein the PEGylated emulsifier has a molecular weight ranging from about 1000 to about 5000.

29. The bilayer nanovesicle of claim 27, wherein the PEGylated emulsifier is selected from the group consisting of N-(methoxypolyethylene glycol 5000 carbamoyl)-1,2-dipalmitoyl-sn-glycero-3-phosphatidylethanolamine (MPEG5000-DPPE), 1,2-dimyristoyl-sn-glycero-3-phosphoethanolamine-N-[methoxy(polyethylene glycol)-2000 (DMPE-PEG2000), 1,2-distearoyl-sn-glycero-3-phosphoethanolamine-N-[methoxy(polyethylene glycol)-2000 (DSPE-PEG2000), Polyoxyethylene 40 stearate (PEG40S) and combinations thereof.

30. The bilayer nanovesicle of claim 28 wherein the PEG or PEG-lipid is present in an amount between 1-10 molar % or between 2-7 molar %.

31. (canceled)

32. The bilayer nanovesicle of claim 1, further comprising cholesterol.

33. The bilayer nanovesicle of claim 32, wherein the remaining composition of the bilayer nanovesicle substantially comprises the cholesterol.

34. The bilayer nanovesicle of claim 32, wherein the cholesterol is present in an amount between 1-60 molar %.

35. The bilayer nanovesicle of claim 1, wherein the bilayer nanovesicle is substantially spherical.

36. The bilayer nanovesicle of claim 1, wherein the bilayer nanovesicle is between about 70-120 nm in diameter.

37. The bilayer nanovesicle of claims 36, wherein the bilayer nanovesicle is between about 90-100 nm in diameter.

38. The bilayer nanovesicle of claim 1, wherein the porphyrin-phospholipid conjugate comprises a metal chelated therein, optionally a radioisotope of a metal.

39. A composition comprising the bilayer nanovesicles of claim 1 in a buffer.

40. A method of performing photodynamic therapy to a target area on a subject, comprising:
a. providing the composition of claim 1;
b. administering the composition to the subject; and
c. irradiating the target area with light of a wavelength that excites the composition to create radicals and/or reactive oxygen species.

41. A method of imaging a target area in a subject, comprising:
a. providing the composition of claim 1;
b. administering the composition to the subject; and
c. measuring and/or detecting fluorescence or photoacoustic signal at the target area.

42. A method of delivering a radioisotope to a subject comprising:
a. providing the composition of claim 1, wherein the bilayer nanovesicle has a radioisotope chelated therein; and
b. administering the composition to the subject.

43. (canceled)

44. (canceled)

45. (canceled)
